(19) United States
(12) Patent Application Publication
(10) Pub. No.: US 2008/0311614 A1
(43) Pub. Date: Dec. 18, 2008

(54) METHODS FOR MEASURING PH IN A SMALL-SCALE CELL CULTURE SYSTEM AND PREDICTING PERFORMANCE OF CELLS IN A LARGE-SCALE CULTURE SYSTEM

(75) Inventor: Gene W. Lee, Chelmsford, MA (US)

Correspondence Address: FITZPATRICK CELLA (WYETH), 30 ROCKEFELLER PLAZA, NEW YORK, NY, 10112-3800, US

(73) Assignee: WYETH, Madison, NJ (US)

(21) Appl. No.: 12/139,174

(22) Filed: Jun. 13, 2008

Related U.S. Application Data
(60) Provisional application No. 60/944,276, filed on Jun. 15, 2007.

(51) Int. Cl. C12Q 1/02

(52) U.S. Cl. 435/29

(57) ABSTRACT

The present invention is directed to methods/systems for measuring the pH of a cell culture medium in a small-scale system utilizing a pH-sensitive dye. The present invention is also directed to methods for predicting the performance of cells in a large-scale culture system.

### CROSS-REFERENCE TO RELATED APPLICATIONS

This application claims the benefit of U.S. Provisional Application No. 60/944,276, filed Jun. 15, 2007, which is incorporated herein by reference in its entirety.

### BACKGROUND OF THE INVENTION

1. Field of the Invention

The present invention is directed to methods for measuring the pH of a cell culture medium in a small-scale culture system. The present invention is also directed to methods for predicting the performance of cells in a large-scale culture system.

2. Related Background Art

Traditional cell cultivation process development involves the screening of large numbers of cell lines in vessels such as shake flask cultures. Further testing of successful candidates can then be performed in another type of vessel, e.g., full-scale bioreactors. The need to carry out numerous development cultivations has prompted the advance of small-scale culture systems, e.g., miniature bioreactors, which offer a high-throughput solution to process development.

Generally speaking, as bioreactors increase in size/scale, more process information is available due to superior monitoring and control systems (Betts and Baganz (2006) “Miniature bioreactors: current practices and future opportunities,” Microbial Cell Factories 5:21). While more capable of high-throughput operations than their full-scale counterparts, small-scale culture systems are generally less instrumented and often have limited opportunity for offline sampling due to the relatively small volumes of the cultures. These constraints result in a trade-off between information content (e.g., in terms of data quality and quantity) and experimental throughput. The most common undesirable consequences of this trade-off are low cell densities and poor cell viability.

The key variables that affect cell growth are: (1) dissolved oxygen, (2) pH, (3) temperature, and (4) availability of nutrients in the culture media. In small-scale systems, the cultures can be placed in an incubator programmed to maintain a particular temperature. The availability of nutrients in the media can be managed by supplementing the culture media with appropriate ingredients. Dissolved oxygen levels can be manipulated by improving the transfer of gases across the air/liquid interface. It is far more difficult, however, to control pH in small-scale systems.

In full-scale bioreactors, pH can be set to a predetermined value and monitored online. Online pH monitoring is not, however, currently available for most small-scale systems. Historically, buffered media was used in an attempt to control pH in small-scale systems. Proper adjustment of cell culture pH, however, requires accurate measurement of the cell culture pH immediately prior to adjustment in most situations. Additionally, for high-throughput assessment, the ability to quickly monitor the pH of a large number of cell cultures simultaneously is critical.

It has been shown that nonbioreactor cell cultures, e.g., small-scale culture systems, that are manually adjusted for pH exhibit cell growth, viability, and productivity behaviors that are superior to nonbioreactor cell cultures that are not adjusted for pH. This is because the addition (e.g., manual addition) of a base, e.g., sodium bicarbonate, can overcome the inhibition of growth that results from the acidification of the cell culture medium by, e.g., metabolism of glucose and secretion of lactate.

Offline instruments, such as blood-gas analyzers, metabolite analyzers, pH meters, etc., have been employed to measure cell culture pH for subsequent manual adjustment. While these instruments may be useful for measuring pH in a small number of cultures, they are inconvenient and time-consuming for large numbers of samples. Additionally, because these methods are not designed to measure large numbers of samples simultaneously, cultures must be inefficiently measured one (or only a few) at a time. Moreover, such methods require a relatively large volume of sample for measurement, which is not compatible with the relatively small working volume of small-scale culture systems. Therefore, there is a need for a method and system to efficiently measure pH in a high-throughput small-scale system.

Instrumented shake flasks designed to measure and potentially control pH and dissolved oxygen levels were recently introduced (Betts and Baganz, supra). For example, it has been shown that both pH and dissolved oxygen can be measured using a ruthenium oxide dye that quantifiably fluoresces in the presence of hydrogen ions or oxygen, respectively, when excited with an LED lamp (see, e.g., Betts and Baganz, supra). In this example, the dye can either be incorporated into a patch and adhered inside a flask or coated onto the tip of a fiber optic-linked probe and immersed into the culture of interest.

The advent of spintubes for small-scale process development cultivations offers several advantages over many other vessels useful in small-scale culture systems. Spintubes are modified 50 ml conical tubes, comprising filtered caps for sterile aeration of the culture. They may be mounted, e.g., on a rotating orbital shaker that can be placed in an incubator (DeJesus et al. (2004) “Tubespin Satellites: A Fast Track Approach for Process Development with Animal Cells Using Shaking Technology,” Biochem. Engineer. J. 17:217-23). Culture volumes can range from about 5 ml or less to about 35 ml per tube. Spintubes can be agitated at high speeds to promote efficient gas exchange, and have been shown to support high cell density growth of mammalian cells (Stettler et al. (2007) “1000 Non-instrumented Bioreactors in a Week,” Cell Technology for Cell Products (Proceedings of the 19^{th} ESACT Meeting, Harrogate, UK), pp. 489-95, Rodney Smith, ed., Springer, The Netherlands). Additionally, these vessels greatly increase the number of cultures that can be evaluated and manipulated by a single operator, relative to other small-scale culture vessels (e.g., shake flasks), because they take up significantly less space. These vessels also exhibit some advantages over smaller volume cell culture vessels as they can support larger volumes for cell culture sampling and analysis.

The use of spintubes allows for high-throughput assessment of culture conditions with relatively large volume cultures that have a low evaporation rate. Until now, however, offline analysis of spintube cultures, e.g., to assess pH, has been carried out using entire tubes on a sacrificial basis. No method has been developed to evaluate culture parameters, such as pH, by evaluating only a small portion of the culture. The development of a simple pH measurement assay applicable for use with spintubes (and other vessels useful for small-scale culture systems) would enable rapid assessment of, e.g., multiple cell culture samples.

Many dyes exhibit colorimetric properties or different fluorescent properties at varying pHs. Examples of such pH-sensitive dyes include, but are not limited to, phenol red, litmus, fluorescein, phenolphthalein, BCECF, carboxy-SNARF, HPTS, carboxy-SNAFL (carboxyseminapthofluorescein), and 5,6-carboxyfluorescein. Some of these dyes, such as carboxy-SNARF-1 (carboxyseminaphthorhodafluor-1) and HPTS (8-hydroxypyrene-1,3,6-trisulphonic acid), are in forms that do not cross cellular membranes, thus preventing cellular uptake. Because of this feature, these dyes may be useful for direct measurement of the pH of cell culture media.

A need exists for the development of a small-scale cell culture system that provides a simple, robust, and efficient method to measure and subsequently control pH in order to closely resemble the conditions and environment present in a full-scale bioreactor.

### SUMMARY OF THE INVENTION

The present invention addresses the above-described problems to provide a method for simultaneous measurement of pH in a large numbers of samples using a relatively small volume of cell culture, thereby providing a method for predicting the performance of cells in a large-scale system.

In one embodiment, the present invention provides a method for measuring the pH of cell culture medium in a small-scale culture system, comprising culturing cells in cell culture fluid/medium in a first cell culture vessel; withdrawing, at least one time, a quantity of the cell culture medium from the first cell culture vessel; placing the withdrawn quantity of cell culture medium into a second vessel (e.g., an assay plate); contacting the withdrawn quantity of cell culture medium with a pH-sensitive dye; and measuring the pH of the withdrawn cell culture medium.

In another embodiment, the present invention provides a method for measuring the pH of cell culture medium in a small-scale culture system, comprising culturing cells in cell culture fluid/medium in a cell culture vessel, contacting the cell culture medium with a pH-sensitive dye, and measuring the pH of the cell culture medium.

In an additional embodiment, the present invention provides a method for predicting the performance of cells in a large-scale culture system, comprising culturing cells in cell culture fluid/medium in a first cell culture vessel; withdrawing, at least one time, a quantity of the cell culture medium from the first cell culture vessel; placing the withdrawn quantity of cell culture medium into a second vessel (e.g., an assay plate); contacting the withdrawn quantity of cell culture medium with a pH-sensitive dye; measuring the pH of the withdrawn cell culture medium; optionally adjusting the pH of the cell culture medium in the first cell culture vessel; and predicting the performance of the cells in a large-scale culture system.

In a further embodiment, the present the present invention provides a method for predicting the performance of cells in a large-scale culture system, comprising culturing cells in cell culture fluid/medium in a cell culture vessel; contacting the cell culture medium with a pH-sensitive dye; measuring the pH of the cell culture medium; optionally adjusting the pH of the cell culture medium; and predicting the performance of the cells in a large-scale culture system.

In yet another embodiment, the present invention provides a small-scale cell culture system providing a means for measuring cell culture pH, comprising cells cultured in cell culture medium in a first cell culture vessel, a means for withdrawing a quantity of the cell culture medium and placing the withdrawn quantity of cell culture medium into a second vessel, a means for contacting the withdrawn quantity of cell culture medium with a pH-sensitive dye, and a means for measuring the pH of the contacted medium.

The method of the present invention is rapid, which permits cultures to be sampled, measured for pH, adjusted for pH (if necessary), and returned to a temperature-controlled environment in a relatively short period of time. Additionally, the method and system of the present invention are relatively inexpensive, and utilize equipment and instrumentation found in most cell culture laboratories. The data presented herein show that cells cultured according to the present invention display growth and viability profiles that are comparable to cells cultured in full-scale bioreactors. Thus, the present invention also provides a method for predicting the performance of cells in a large-scale culture systems. Moreover, the data demonstrate that the present invention is a novel and effective way to identify the top performing cell lines or clones used for large-scale cell culture-based processes.

### BRIEF DESCRIPTION OF THE DRAWINGS

FIG. 1 shows a standard curve for carboxy-SNARF using phosphate-buffered saline (PBS).

FIG. 2 shows the pH of PBS standards measured at varying times of incubation using carboxy-SNARF.

FIG. 3 shows a comparison of pH measurements using either carboxy-SNARF (SNARF) or a blood gas analyzer (BGA).

FIG. 4 shows the growth over time (days in culture) of cultures of mAb-producing cells, wherein the pH of the culture was either adjusted or not adjusted.

FIG. 5 shows mAb production by cultured cells, wherein the pH of the culture was either adjusted or not adjusted.

FIG. 6 shows a comparison of the analysis of antibody titer (mg/L) of 24 cell line clones cultured either with or without pH adjustment.

FIG. 7 shows a comparison of recombinant mAb production by cell lines cultured according to the present invention in a 10 mL small-scale culture system or in a 2 L bioreactor.

FIG. 8 shows a comparison of recombinant mAb production in a lead cell line cultured according to the present invention in a 10 mL small-scale culture system or in varying scale bioreactors (at 2 L, 190 L, and 6000 L scales).

FIG. 9 shows a comparison of either mAb production (Ab1-Ab4) or fusion protein production (FP1 and FP2) in a lead cell line cultured according to the present invention in a 10 mL small-scale culture system or in varying scale bioreactors (at 2 L, 190 L, 500 L, and 6000 L scales).

### DETAILED DESCRIPTION OF THE INVENTION

The various embodiments of the present invention enable the performance of cell lines grown under the conditions described to closely match the performance of the same cell lines when grown in a controlled, full-scale, stirred-tank bioreactor. Given the small size/scale and simplicity of the method and system described, the present invention can be used either in conjunction with or in the place of bioreactors for cell-line screening, process development, medium development, or process confirmation. Moreover, the invention is compatible with assessing large numbers of cultures simultaneously, greatly expanding the capability of a cell culture lab. The ability to assess large numbers of cultures may be useful for many research endeavors involving cell culture, including, but not limited to, cell line screening or factorial design experiments.

The methods and system described herein may also offer advantages over conventional bioreactors. The scale-up of cell culture to provide a suitable volume of cell culture to inoculate a single standard benchtop bioreactor (i.e., 1 or 2 liters of working volume) can take several days or weeks, depending on the growth rate of the cells. In contrast, minimal or practically no scale-up time is required to support the inoculation of a single spintube culture (10 milliliters of working volume), or alternatively many spintubes can be inoculated with a larger volume of scaled-up inoculum culture.

One aspect of the present invention is directed to a method for measuring the pH of a cell culture medium in a small-scale culture system, comprising culturing cells in cell culture medium in a first cell culture vessel; at least once (for example, periodically) withdrawing a quantity of the cell culture medium; placing the withdrawn quantity of cell culture medium into a second vessel (for example, an individual well in a 96-well plate); contacting the withdrawn quantity of cell culture medium with a pH-sensitive dye (for example, a fluorescent pH-sensitive dye) in the second vessel; and measuring the pH of the withdrawn cell culture medium (for example, by using a fluorescent plate reader). One of skill in the art will recognize that the pH-sensitive dye can be introduced to the second vessel either before or after the withdrawn quantity of cell culture medium is placed into the vessel.

The present invention is also directed to a method for predicting the performance of cells in a large-scale culture system, comprising culturing cells in cell culture medium in a first cell culture vessel; at least once (for example, periodically) withdrawing a quantity of the cell culture medium; placing the withdrawn quantity of cell culture medium into a second vessel (for example, an individual well in a 96-well plate); contacting the withdrawn quantity of cell culture medium with a pH-sensitive dye (for example, a fluorescent pH-sensitive dye) in the second vessel; measuring the pH of the withdrawn cell culture medium (for example, by using a fluorescent plate reader), optionally adjusting the pH of the cell culture medium in the first cell culture vessel (for example, by using an acid or a base); and predicting the performance of the cells in a large-scale culture system.

The present invention is further directed to a small-scale cell culture system providing a means for measuring cell culture pH, comprising cells cultured in cell culture medium in a first cell culture vessel, a means for withdrawing a quantity of the cell culture medium and placing the withdrawn quantity of cell culture medium into a second vessel, a means for contacting the withdrawn quantity of cell culture medium with a pH-sensitive dye, and a means for measuring the pH of the contacted medium.

The first cell culture vessel, which is in a small-scale culture system, may be any suitable culture vessel lacking online controls, including but not limited to conical tubes (e.g., conical tubes with modified caps, e.g., spintubes), shake flasks, spinner flasks, and multi-well plates, e.g., 12-well and 24-well plates. In a preferred embodiment of the invention, the first cell culture vessel is a conical tube; especially preferred is a 50 ml conical tube having a cap (e.g., a top, lid, or other form of covering) that allows for the sterile exchange of gases, e.g., a spintube.

The cell culture fluid/medium may comprise any type of fluid/medium suitable for culturing the cells of interest, e.g., medium that is formulated to support growth of eukaryotic or prokaryotic cells in vitro, that also supports the use of a pH-sensitive dye; such fluids/media are well known to one of skill in the art. Examples include, but are not limited to, Minimal Essential Media (MEM), Dulbecco's MEM (DMEM), and AIM V, supplemented with serum (up to about 20%) and other appropriate ingredients, such as antibiotics. Specific nonlimiting examples include cell culture media comprising whole DMEM media (e.g., DMEM, high glucose with 10% fetal bovine serum, 1% nonessential amino acids, and 1% penicillin-streptomycin). In addition, the method of the invention can be applied to any fluid/aqueous solution, including non-cell culture samples. For example, the method can be used to determine the pH of a large number of biological samples, buffers, solutions, etc. simultaneously.

Cells may be cultured under any conditions appropriate to the cells of interest, as would be known by one of skill in the art. For example, cells in culture medium may be cultured at 37° C. or 31° C. with 5% CO₂. The temperature of the culture vessels may be controlled by any procedure or device known in the art, including but not limited to incubators such as Forma CO2 Incubator Model 3950 or 3956 (Thermo-Forma, Marietta, Ohio) or Kuhner Model ISFI-W or -X (Kuhner AG, Basel, Switzerland). One of ordinary skill will recognize the need to manipulate the gas exchange between the atmosphere in, e.g., the incubator, and the culture medium, in order to obtain the desired level of dissolved oxygen; such exchange can occur, e.g., by passive transfer at the interface of the atmosphere and the culture, or by agitation of the culture. In a preferred embodiment, cell culture aeration is accomplished with rapid agitation of cultures in spintubes; several spintubes can be agitated simultaneously in a rack housed in an incubator.

The volume of cell culture medium may range from about 200 μl or less to about 20 L or more; more preferably from about 1 ml to about 1 L; especially preferable is a volume of about 10 ml (e.g., cultured in a 50 ml conical tube, e.g., a spintube). Generally, “small-scale cell culture,” “small-scale culture system,” and the like refer to a volume of about 2 L or less, although the methods of the present invention may be employed with any volume of fluid, e.g., cell culture medium.

The quantity of cell culture medium withdrawn from the first cell culture vessel may range from about 25 μl to about 25 ml or greater, but is preferably about 50 μl to about 300 μl. The cell culture medium may be withdrawn one time, once per day, once every other day, multiple times per day, or any other time interval deemed adequate or necessary, which may depend upon factors such as rate of cell growth and type of culture media. In a preferred embodiment of the invention, the cell culture medium is withdrawn daily in a volume of about 200 μl.

The second vessel may be any suitable vessel, lacking online pH controls, in which the pH of the cell culture medium may be measured using a pH-sensitive dye, such as a calorimetric or fluorescent pH-sensitive dye. For example, any assay plate, e.g., any multi-well plate, such as a 24-, 96- or 384-well plate that may be placed in a plate reader is suitable. In a preferred embodiment of the invention, the second vessel is a 96-well microtiter plate.

In another embodiment, the invention provides a method of measuring the pH of cell culture medium comprising only one vessel. Thus, the present invention provides a method for measuring the pH of cell culture medium in a small-scale culture system, comprising culturing cells in cell culture medium in a cell culture vessel, contacting the cell culture medium with a pH-sensitive dye, and measuring the pH of the cell culture medium. In some preferred embodiments, the vessel may be any multi-well plate, e.g., a 24-, 96- or 384-well plate that may be placed in a plate reader for measurement of pH. Such embodiments of the invention may be useful when one or more of the wells (or some other vessels) are appropriately to be sacrificed.

An additional aspect of the present the present invention is directed to a method for predicting the performance of cells in a large-scale culture system, comprising culturing cells in cell culture fluid/medium in a cell culture vessel (for example, an individual well in a 96-well plate); contacting the cell culture medium with a pH-sensitive dye (for example, a fluorescent pH-sensitive dye), measuring the pH of the cell culture medium (for example, by using a fluorescent plate reader); optionally adjusting the pH of the cell culture medium (for example, by using an acid or a base); and predicting the performance of the cells in a large-scale culture system.

Any pH-sensitive dye that is not taken up by the cultured cells may be used in the present invention to measure the pH of the cell culture medium. At least one preferred embodiment of the present invention uses a calorimetric pH-sensitive dye to measure pH. At least one other preferred embodiment of the present invention uses a fluorescent pH-sensitive dye to measure pH; especially preferred is carboxy-SNARF or HPTS. For example, carboxy-SNARF-1 is a dye that does not cross the cell membrane, and is useful for measurement of the pH of cell culture medium/fluid without interference from measurement of intracellular pH (carboxy-SNARF-1 is cell-membrane impermeable, as opposed to its ester form, e.g., carboxy-SNARF-1-acetoxymethyl ester (see, e.g., Qian et al. (1997) Am. J. Physiol. 273:C1783-92)).

The pH of the cell culture medium may be quantified by any instrument with the ability to detect pH-mediated changes in pH-sensitive dyes, as would be known by one of skill in the art. Instruments include those that measure fluorescence and those that measure color, e.g., spectrophotometers. Nonlimiting examples of automated devises for such measurements include plate readers such as SPECTRAmax Gemini EM and SPECTRAmax M2 fluorescent plate readers (Molecular Devices, Sunnyvale, Calif.), Packard LumiCount microplate luminometer (Packard Instruments, Meriden, Conn.), and Cytofluor II Fluorescent Microplate Reader (Perseptive Biosystems, Framingham, Mass.).

After the pH of the cell culture medium (either withdrawn from the first cell culture vessel or in the one cell culture vessel) is determined, the cell culture medium (either remaining in the first cell culture vessel or in the one cell culture vessel) may be adjusted, if necessary, to a desired value. This pH adjustment may be accomplished by any means known in the art, such as adding a base or an acid to the cell culture medium. In a preferred embodiment of the invention, the pH of the cell culture medium in the first cell culture vessel is adjusted by adding either a sodium bicarbonate solution or a lactic acid solution, depending upon the pH value determined by method of the invention. The amount of base or acid to be added to the cell culture medium to adjust the pH may be easily calculated by one of skill in the art.

The ability to adjust the pH of cell culture medium, as needed, in a small-scale culture system enables conditions that mimic large-scale culture systems. Thus, cell performance in the small-scale culture system is predictive of the cell performance in a large-scale culture system under the same conditions. Generally, “performance” refers to the growth, viability, and productivity characteristics of cells. Since the present invention provides for the simultaneous measurement of pH in a large number of samples from a small-scale culture system, the effects of several variables on cell performance in small-scale systems can now be tested to determine which variable, such as, but not limited to, cell line, medium component, and/or processing methods, would provide the best performance. Based on the variable being tested, one skilled in the art would know what performance characteristic would be desired, such as, but not limited to, rapid growth, longer cell viability, and/or increased production.

The entire contents of all references, patents, and patent applications cited throughout this application are hereby incorporated by reference herein.

### EXAMPLES

The Examples do not include detailed descriptions of conventional methods, such as methods employed in the culturing of cells. Such methods are well known to those of ordinary skill in the art.

### Example 1

### Materials and Methods

### Example 1.1

### Cell Lines

All recombinant cell lines were developed using CHO host cells. Product gene expression is maintained through selection for DHFR expression by culturing in the presence of methotrexate. Candidate cell lines were adapted to serum-free suspension culture prior to small-scale fed batch evaluation.

### Example 1.2

### Fed-Batch Culture

For small-scale evaluations, duplicate or triplicate 10 ml cultures were evaluated for each cell line in a Kuhner shaking incubator (Model ISF1-W; Kuhner AG, Basel, Switzerland), using TubeSpin “disposable bioreactors” (“spintubes”) (TPP AG, Trasadingen, Switzerland). The incubator was run at 7% CO₂, at 37° C. or 31° C. The base and feed media used in the method and system of the present invention were identical to those used in the bioreactors. The base media and the feed media used in these experiments were specifically developed to support high cell densities of recombinant CHO cell lines in fed-batch culture. The bioreactors were operated with dO₂, temperature and pH controlled to setpoints of 30%, 37° C./31° C., and 7.0, respectively.

### Example 1.3

### pH Measurement

To measure cell culture pH, a small volume of culture (about 200 μl) was withdrawn daily from each spintube and placed into a well of a 96-well microtiter plate. Each well had been prealiquotted with about 5 μl of carboxy-SNARF solution (Invitrogen). The plate was then run on a fluorescent plate reader (SpectraMax M2, Molecular Devices, Sunnyvale, Calif.), and the ratio of the fluorescent signals at 580 nm and 640 nm wavelengths was determined. A three-point standard curve was run on each plate to determine the pH of the sampled cultures. Alternatively, the pH of cell culture samples was measured using a blood gas analyzer (CIBA-Corning BGA Model 248, Walpole, Mass.). The culture pH was adjusted to a predetermined setpoint, if necessary, through the addition of a volume of a 1M sodium bicarbonate solution. The base was added directly to the cell culture, such that the final pH of the culture was pH 7.3. This pH setpoint may be changed to 7.2, 7.1, 7.0 etc. The pH of a bicarbonate-containing cell culture medium may also be adjusted by changing the CO₂ level in an incubator.

### Example 2

### pH Measurement with Carboxy-SNARF

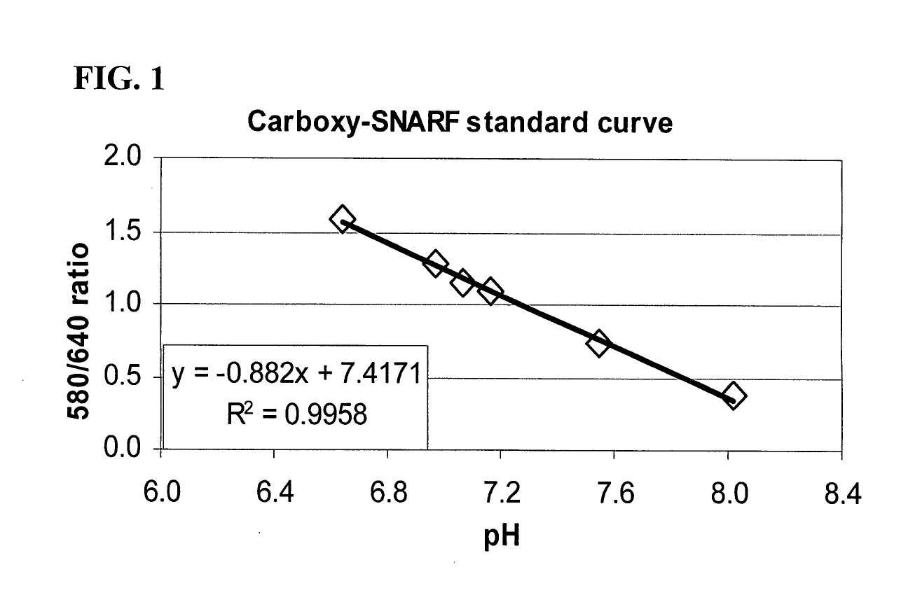

FIG. 1 shows a standard curve for carboxy-SNARF using phosphate-buffered saline (PBS). PBS was adjusted with HCl or NaOH, and the pH was measured using a standard laboratory pH meter. 200 μl of the pH-adjusted PBS solutions was incubated for approximately two minutes at room temperature with 5 μl of a 1 mM carboxy-SNARF solution in a 96-well plate, and the plate was read on a fluorescent plate reader at an excitation wavelength of 490 nm and dual emission wavelengths at 580 and 640 nm. The standard curve demonstrates that the ratio of emissions at these wavelengths can be used reliably to determine the pH of an unknown sample using this method. These data demonstrate that carboxy-SNARF exhibits a linear response over a range of pH values that would typically be encountered in cell cultures.

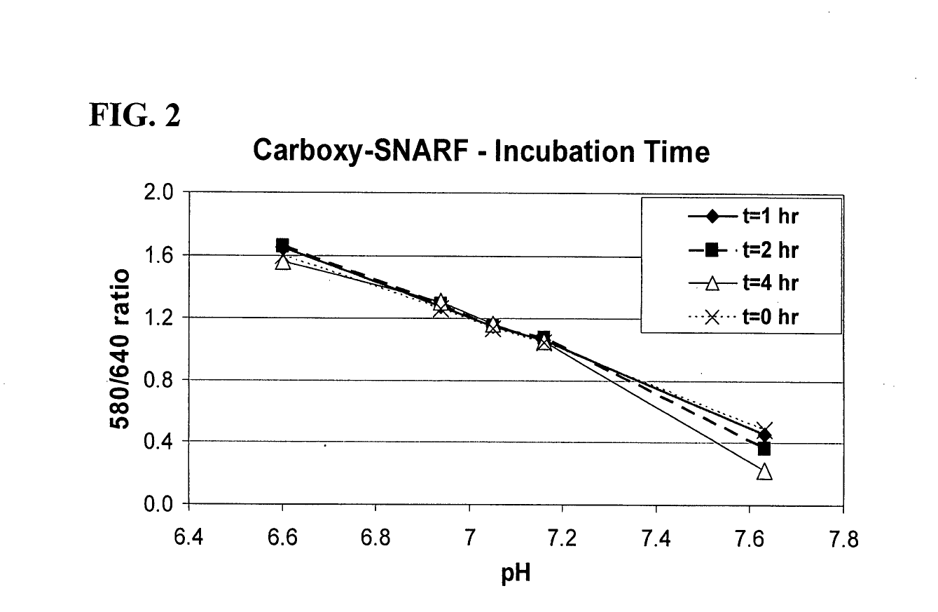

FIG. 2 shows the pH of PBS standards measured at varying times using carboxy-SNARF. PBS standards (200 μl) were incubated for varying times with 5 μl of carboxy-SNARF in a 96-well plate. pH was then measured either immediately after this incubation (t=0 hr), or after varying time periods of incubation at room temperature (at 1 hr, 2 hr, or 4 hr) using a fluorescent plate reader. These data show that the carboxy-SNARF signal remains stable over several hours, thus making it compatible with measuring large numbers of samples.

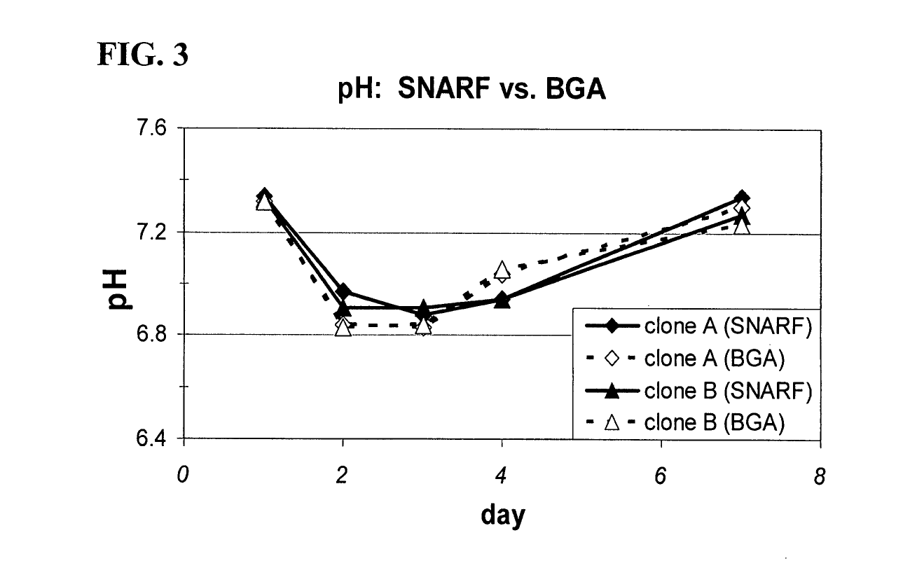

FIG. 3 shows a comparison of pH measurements using either carboxy-SNARF or a blood gas analyzer (BGA). Two CHO cell lines (clone A and clone B) were evaluated in a fed-batch assay as described herein. On days 1, 2, 3, 4, and 7, the pH of the cultures was measured using either carboxy-SNARF (solid lines) or a blood gas analyzer (dotted lines). These data show that measurements of the pH of culture samples by use of carboxy-SNARF are very similar to measurements made using a conventional offline method.

### Example 3

### Effects of pH Adjustment

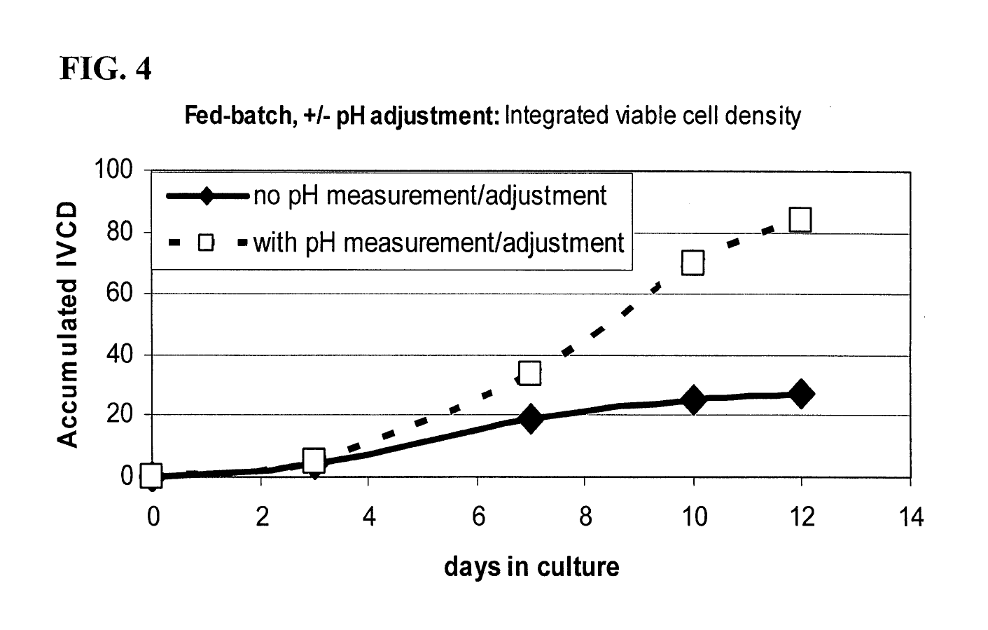

FIG. 4 shows the growth over time of cultures of mAb-producing cells (accumulated IVCD (integrated viable cell density)), wherein the pH of the culture either was not measured and adjusted (solid diamonds, solid line) or was measured and adjusted, if adjustment was necessary (open squares, dotted line). An equivalent number of cells were seeded for the two conditions shown, and all other process parameters were identical. Cells were seeded in base media and fed with feed media on days 3, 7 and 10. Cultures were shifted from 37° C. to 31° C. on day 3. Cell density measurements were made using the Guava PCA instrument (Guava Technologies, Hayward, Calif.). The pH of the cultures was measured on days 1, 2, 3, 7, and 10.

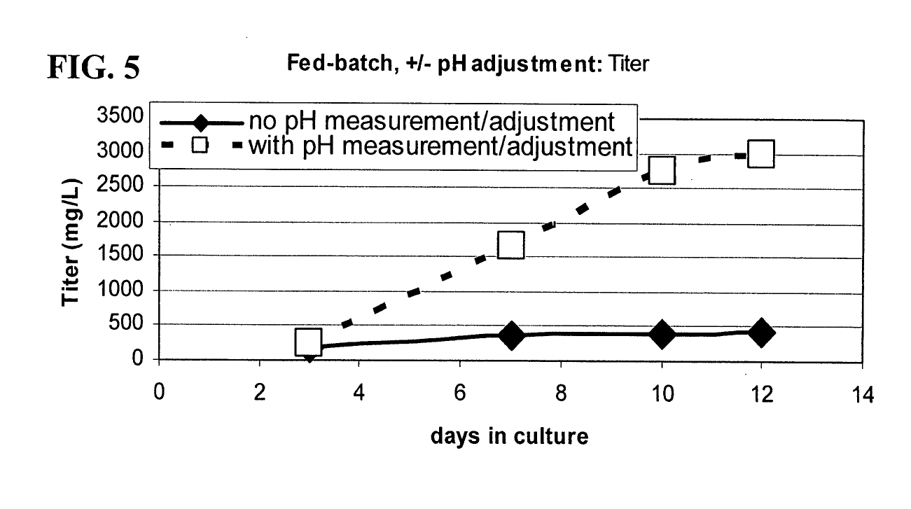

FIG. 5 shows mAb production by cultured cells, wherein the pH of the culture either was not measured and adjusted (solid diamonds, solid line) or was measured and adjusted, if adjustment was necessary (open squares, dotted line). Experimental procedures were the same as described for FIG. 4. Titer (product concentration) was assessed by Protein A-HPLC measurement.

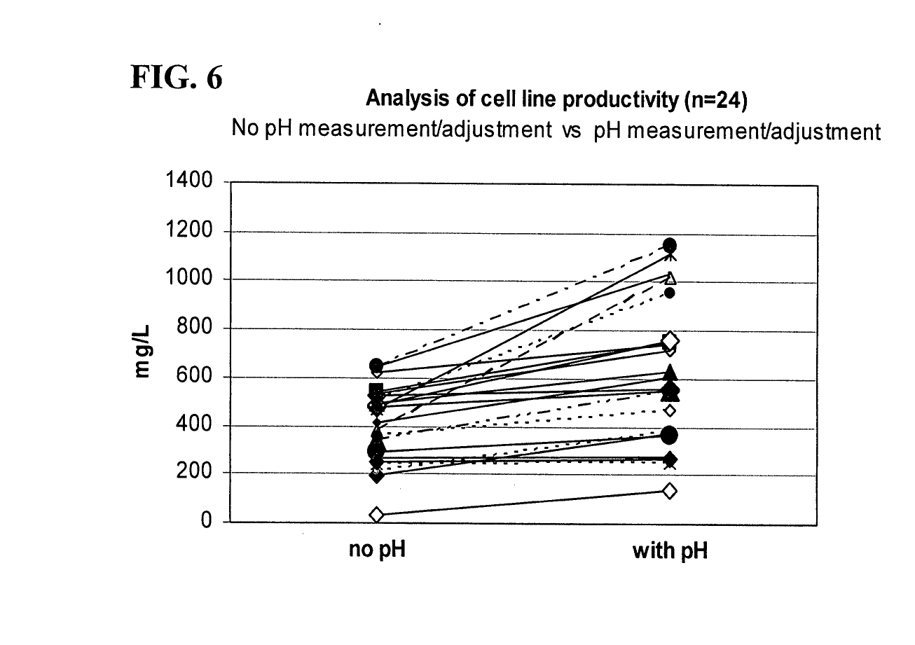

FIG. 6 shows a comparison of the analysis of antibody titer (mg/L) of 24 mAb-producing cell line clones cultured either with pH adjustment (with pH)) or without pH adjustment (no pH). Cell lines were evaluated in a fed-batch production assay, using conditions essentially as described for FIGS. 4 and 5. The data demonstrate a general trend toward improved antibody titer with pH adjustment.

### Example 4

### Comparison of Small-scale System and Bioreactor

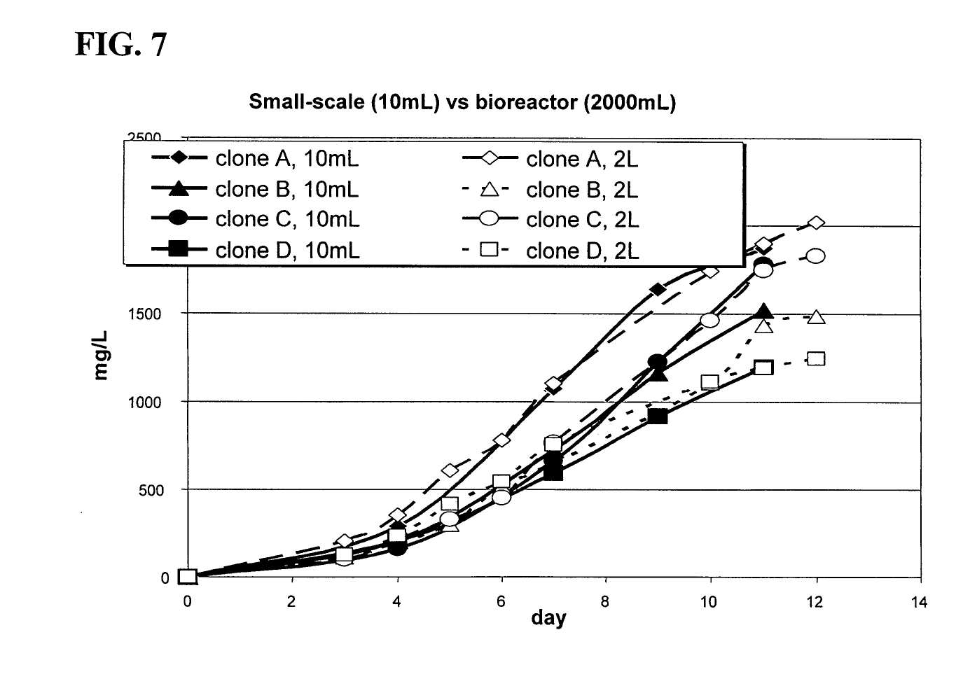

FIG. 7 shows a comparison of recombinant mAb production by cell lines cultured either according to the present invention (small-scale culture system) or in a full-scale bioreactor. Four CHO cell lines (clones A-D) that produce recombinant mAbs were evaluated in a fed-batch assay as described above, either in the small-scale system of the present invention (10 ml in spintubes) or in 2 L bioreactors. Product titer was determined by Protein A HPLC. Experimental conditions for the spintubes was essentially as described fro FIG. 4. In the 2 L bioreactors, cells were inoculated into the same base media at the same cell density as the spintube cultures, and were fed with the same feed media. Cultures in the bioreactors were shifted from 37° C. to 31° C. on approximately day 3. The pH setpoint in the bioreactor was pH 7.0.

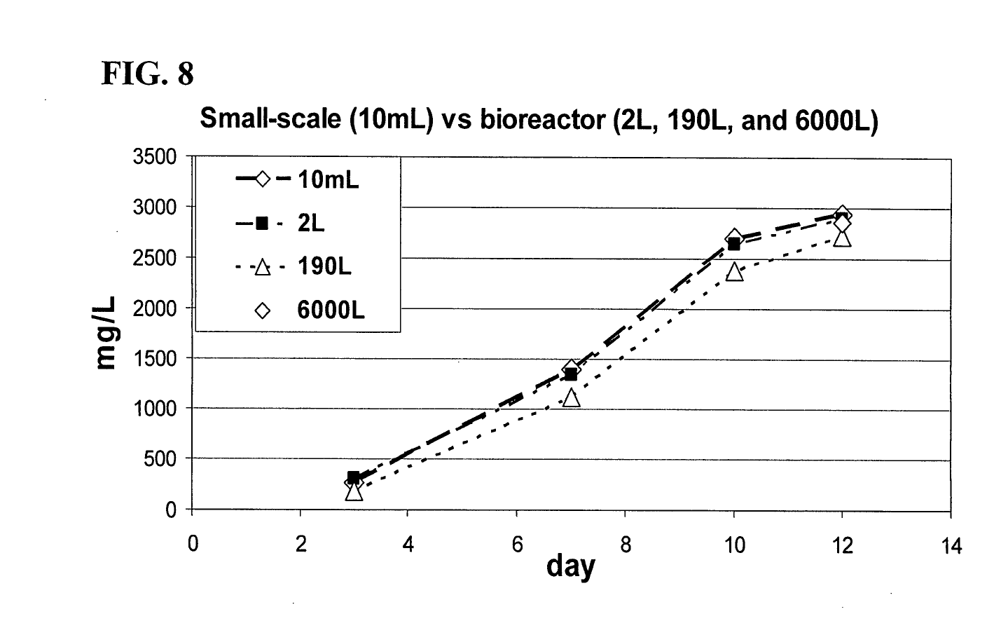

FIG. 8 shows a comparison of recombinant mAb production in a cell line producing a therapeutic anti-cytokine mAb cultured according to the present invention or in varying scale bioreactors. The cell line, in an early-stage cell line-development program, was evaluated in a fed-batch assay in the small-scale system of the present invention (10 ml in spintubes) or in bioreactors at 2 L, 190 L and 6000 L scales. The fed-batch culture conditions and parameters were identical at all bioreactor scales.

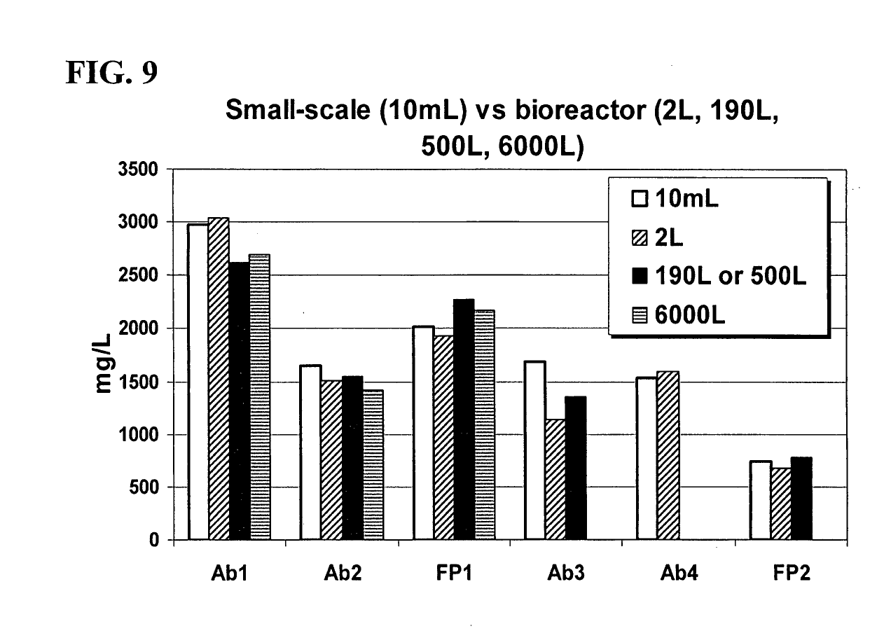

FIG. 9 shows a comparison of either mAb production (Ab1-Ab4) or fusion protein expression (FP1 and FP2) in cell lines cultured according to the present invention or in varying scale bioreactors. The cell lines were assessed in either the small-scale system of the present invention (10 ml in spintubes) or in bioreactors at 2 L, 190 L, 500 L, and 6000 L scales.

According to the present invention, the pH of cell cultures may be adjusted by the addition of a titrant (e.g., either a base or an acid), such as sodium bicarbonate. The data presented herein demonstrate that the overall performance of pH-adjusted cell lines cultured according to the present invention is markedly better than cultures not pH-adjusted, and that cells cultured according to the present invention display growth and viability profiles that are comparable to cells cultured in full-scale bioreactors. Moreover, individual cell lines respond differently to a pH-adjusted system. Therefore, the method and system of the present invention further provides a more effective way to rapidly screen a large number of candidate clones to identify the top process-ready clones (i.e., clones that are predicted to be productive in bioreactors, e.g., large-scale bioreactors). The top clones may then be further evaluated in a smaller number of full-scale bioreactors, if required.

The methods and system of the present invention provides for a cell culture environment that closely approximates that which is present in a full-scale, stirred-tank bioreactor, e.g., including providing the advantage of measuring and, when necessary, adjusting the pH of the cell culture environment/medium. The present invention provides an offline method and system for measuring the pH of cell culture media in small-scale cell culture systems or related systems. The performance of cells lines cultured according to the present invention is indistinguishable from the performance of cell lines cultured in large-scale bioreactors, at least up to the 6000 L scale. The simplicity and flexibility of the method and system of the present invention, coupled with the performance, make this novel method a valuable advance in cell line and process development.

The above embodiments of the present invention have been described for purposes of illustrating how the invention may be made and used and do not in any way limit the invention. Other variations and modifications of the invention and its various aspects will become apparent, after having read this disclosure, to one skilled in the art, and all such variations and modifications are considered to fall within the scope of the invention, which is defined by the appended claims.

What is claimed is:

1. A method for measuring the pH of a cell culture medium in a small-scale culture system, comprising:
culturing cells in the cell culture medium in a first cell culture vessel;
withdrawing, at least one time, a quantity of the cell culture medium from the first cell culture vessel;
placing the withdrawn quantity of cell culture medium into a second vessel;
contacting the withdrawn quantity of cell culture medium with a pH-sensitive dye; and
measuring the pH of the withdrawn cell culture medium.

2. The method of claim 1, wherein the first cell culture vessel is a conical tube.

3. The method of claim 2, wherein the conical tube has a cap that allows for the sterile exchange of gases.

4. The method of claim 3, wherein the conical tube is a spintube.

5. The method of claim 1, wherein the withdrawn quantity of cell culture medium is withdrawn daily.

6. The method of claim 1, wherein the withdrawn quantity of cell culture medium is between about 50 μl and about 300 μl.

7. The method of claim 6, wherein the withdrawn quantity of cell culture medium is about 200 μl.

8. The method of claim 1, where the second vessel is a microtiter plate.

9. The method of claim 8, wherein the microtiter plate is a 96-well plate.

10. The method of claim 1, wherein the pH-sensitive dye is a fluorescent dye.

11. The method of claim 10, wherein the fluorescent dye is carboxy-SNARF.

12. The method of claim 10, wherein the fluorescent dye is HPTS.

13. The method of claim 1, further comprising adjusting the pH of the cell culture medium in the first cell culture vessel.

14. The method of claim 13, wherein the pH is adjusted using a base.

15. The method of claim 14, wherein the base is a sodium bicarbonate solution.

16. The method of claim 13, wherein the pH is adjusted using an acid.

17. The method of claim 16, wherein the acid is a lactic acid solution.

18. A method for predicting the performance of cells in a large-scale culture system, comprising:
measuring the pH of cell culture medium in a small-scale culture system according to the method of claim 1;
optionally adjusting the pH of the cell culture medium in the first cell culture vessel; and
predicting the performance of the cells in a large-scale culture system.

19. A method for measuring pH of cell culture medium in a small-scale culture system, comprising:
culturing cells in cell culture medium in a cell culture vessel;
contacting the cell culture medium with a pH-sensitive dye; and
measuring the pH of the cell culture medium.

20. A method for predicting the performance of cells in a large-scale culture system, comprising:
measuring the pH of cell culture medium in a small-scale culture system according to the method of claim 19;
optionally adjusting the pH of the cell culture medium; and
predicting the performance of the cells in a large-scale culture system.
